(19) United States
(12) Patent Application Publication
(10) Pub. No.: US 2020/0222224 A1
(43) Pub. Date: Jul. 16, 2020

(54) SEXUAL AID DEVICE AND METHOD OF USING THE SAME

(71) Applicant: Shamiron Jacklene Lopez, Bedford Park, IL (US)

(72) Inventor: Shamiron Jacklene Lopez, Bedford Park, IL (US)

(21) Appl. No.: 16/248,968

(22) Filed: Jan. 16, 2019

(51) Int. Cl. A61F 5/41

(52) U.S. Cl. CPC A61F 5/41; A61F 2005/411

(57) ABSTRACT

A penile sleeve for promoting sexual stimulation utilizing crystals during sexual intercourse, and a method of using the same is provided. The penile sleeve incorporates one or more crystals for facilitating vaginal stimulation during sexual intercourse. The crystal or crystals are interchangeably sheathed to the human penis by a comfortable and stimulating silicone-like material, yet the crystal protrudes through a lower opening in the penile sleeve. As a result, the crystal comes in contact with both the penis and vagina during intercourse enabling a shared experience or energy based on the installed crystal. Additionally, the structure of the incorporated crystal supports a penile orientation that assists the user in stimulating the G-spot.

### BACKGROUND OF THE INVENTION

The present invention relates to methods and devices utilizing crystal for increasing human sexual arousal, and more particularly, to methods and devices embodying a penile sleeve incorporating crystals for promoting vaginal stimulation during sexual intercourse.

The G-spot, also called the Gräfenberg spot is characterized as an erogenous area of the vagina that, when stimulated, may lead to strong sexual arousal. Unfortunately, hitting a woman's G-spot is often difficult and can create negative energy while having sexual intercourse.

Other sexual aid devices utilizing sex crystals are not made to assist the penis in hitting the G-spot, only the crystal. Making it less likely to create a stronger bond with one's sex partner. Furthermore, the crystals are very hard and may cause some discomfort, and so, they are primary use is for masturbation.

As can be seen, there is a need for methods and devices embodying a penile sleeve incorporating crystals for promoting vaginal stimulation during sexual intercourse. The crystal is sheathed to the human penis by comfortable and stimulating silicone-like material, yet the crystal protrudes therethrough, helping assist the partner in stimulating the G-spot, while simultaneously comfortably using a crystal to promote sexual simulation.

The sexual aide removably and interchangeably integrates a genuine crystal having a curvature that protrudes through a lower opening in the penile sleeve, so this simultaneous contact between the crystal and both sexual partners can promote shared sexual stimulation and certain moods the user(s) associate with the user's crystal of choice. Moreover, the crystal structurally supports the partner's penis for stimulating the G-spot, while the crystal itself promotes certain shared energies as it comes in contact with both penis and vagina.

### SUMMARY OF THE INVENTION

In one aspect of the present invention, a sexual aid device includes the following: a penile sleeve having an upper portion and a lower portion; the upper portion dimensioned and adapted to be located along an upper portion of a human wearer's erect penis when said penile sleeve is worn; the lower portion dimensioned and adapted to be located along a lower portion of a human wearer's erect penis when said penile sleeve is worn; a plurality of stabilizing straps provided along the upper portion, each adjacent stabilizing strap are spaced apart by an upper void; and a crystal opening provided in the lower portion.

In another aspect of the present invention, the sexual aid device sexual aid device includes the following: a penile sleeve having an upper portion and a lower portion; the upper portion dimensioned and adapted to be located along an upper portion of a human wearer's erect penis when said penile sleeve is worn; the lower portion dimensioned and adapted to be located along a lower portion of a human wearer's erect penis when said penile sleeve is worn; a plurality of stabilizing straps provided along the upper portion, each adjacent stabilizing strap are spaced apart by an upper void, wherein each upper void is dimensioned for a human finger to pass therethrough; a crystal opening provided in the lower portion; one or more crystal straps spanning the crystal opening; a plurality of ribbing disposed along the lower portion; and a crystal dimensioned to be substantially coextensive with the crystal opening, wherein the crystal provides a convex curvature that protrudes through the crystal opening when the crystal is sandwiched between the one or more crystal straps and said erect penis, and the crystal provides a concave curvature opposing the convex curvature, wherein the concave curvature supports the erect penis when the crystal is sandwiched between the one or more crystal straps and said erect penis.

In yet another aspect of the present invention, a method of promoting sexual stimulation utilizing crystals during sexual intercourse includes the following: providing the above-mentioned sexual aid device; and utilizing a human finger through one of the upper voids to pull the penile sleeve over the erect penis so that the crystal is sandwich between both yet a portion of the crystal protrudes through the crystal opening.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a perspective view of an exemplary embodiment of the present invention;

FIG. 3 is a perspective view of an exemplary embodiment of the present invention;

FIG. 4 is an exploded view of an exemplary embodiment of the present invention;

FIG. 5 is a section view of an exemplary embodiment of the present invention, taken along line 5-5 of FIG. 1; and

FIG. 6 is a section view of an exemplary embodiment of the present invention, taken along line 6-6 of FIG. 5.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method for promoting sexual stimulation utilizing crystals during sexual intercourse. The method embodies a penile sleeve incorporating crystals facilitating vaginal stimulation during sexual intercourse. The crystal or crystals are interchangeably sheathed to the human penis by comfortable and stimulating silicone-like material, yet the crystal protrudes through a lower opening in the penile sleeve. As a result, the crystal comes in contact with both the penis and vagina during intercourse enabling a shared experience or energy based on the installed crystal. Additionally, the structure of the incorporated crystal supports a penile orientation that assists the user in stimulating the G-spot.

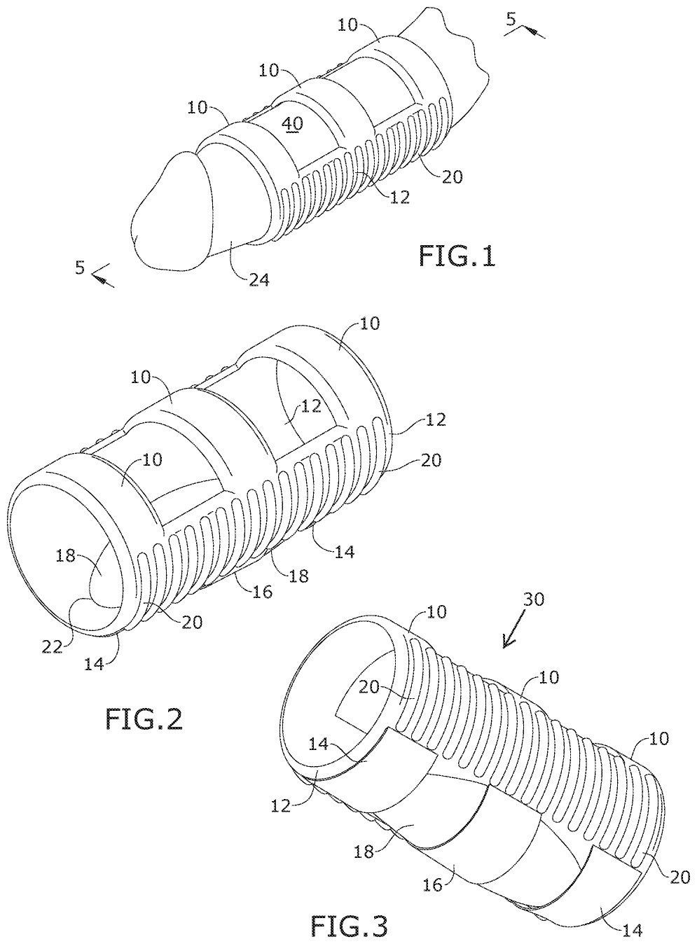

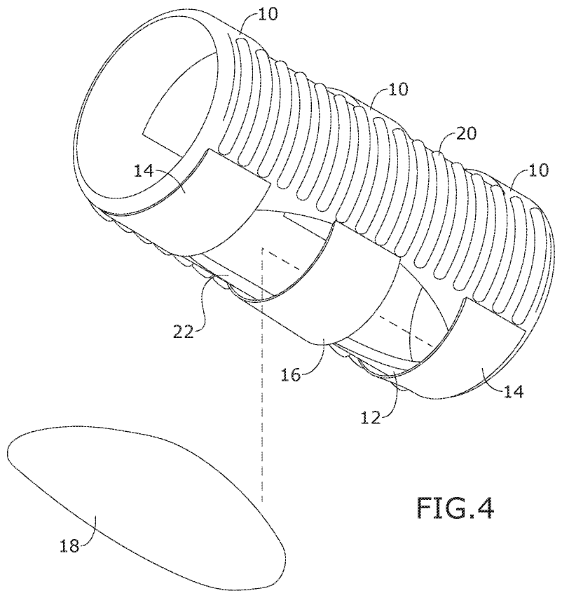

Referring to FIGS. 1 through 6, the present invention may include a penile sleeve 30. The penile sleeve 30 may have an upper portion and a lower portion 12; the upper portion being adapted to be located along an upper portion of a wearer's erect penis 24 when said penile sleeve 30 is worn, while the lower portion 12 is adapted to be located along a lower portion of a wearer's erect penis 24 when said penile sleeve 30 is worn.

The upper portion may provide a plurality of stabilizing straps 10 that will go around the top of the penis 24. The stabilizing straps 10 may be spaced apart and defined by upper voids 40. The stabilizing straps 10 are critical for placing and removing the tight-fitting penile sleeve 30 on a human user.

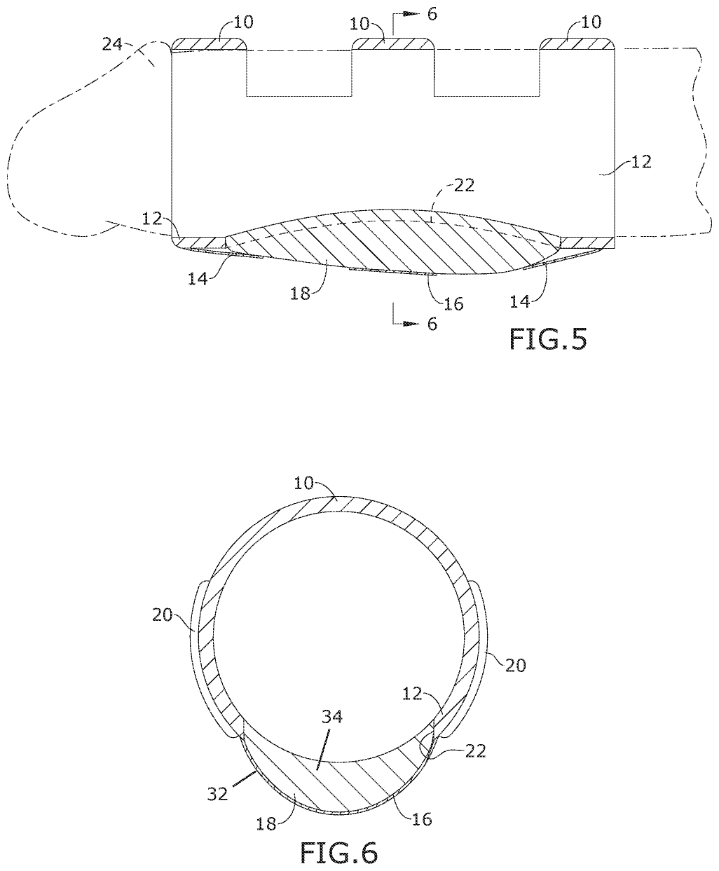

The lower portion 12 may also provide a crystal opening 22. Extending across a portion of the crystal opening 22 may be a central crystal strap 16 and one or more end crystal straps 14. The crystal opening 22 is dimensioned so that one or more crystals 18 may protrude therethrough when the crystal(s) 18 are securely sandwiched between the penis 24 and the crystal straps 16 and 14 during use, as illustrated in FIG. 5. This structure of crystal opening 22 and operatively associated crystal strapping 14 and 16 make the interchangeability of various types of crystal 18 convenient.

Each crystal 18 may have a convex outer curvature 32 and a concaved inner curvature 34 resembling the shape of an empty teaspoon. In certain embodiments, the crystal 18 may have a minimized concaved inner curvature so that its shape resembles a partially-filled teaspoon. In the above-mentioned protruded state, the convex outer curvature 32 may abut internal portions of a human vagina during intercourse, enabling the crystals 18 to release their energy once it comes in contact therewith, while the concaved inner curvature 34 does the same to the user's penis 24.

The crystal 18 secured in the protruded state also may utilize its rigid inner concave curvature to support the penis 24 in a more linear orientation to stimulate the G-spot shape. The size of the crystal 18 will also give more girth to the penis 24 while in use. The lower portion 12 may also provide a plurality of ribbing 20 for sexual excitation purposes.

A method of using the present invention may include the following. The penile sleeve 30 disclosed above may be provided. Once a user has decided they would like a sexual aid device to help stimulate the G-spot and appreciate benefits of using crystals 18 during intercourse, they would just choose which crystal 18 they would like to use for a preferred associated energy. Then the user or their partner may pull the penile sleeve 30 over and around the penis 24 by engaging the stabilizing straps 10 so that the penile sleeve 30 provide some tensions on the penis 24.

Once the penile sleeve 30 is properly on, the wearer can begin intercourse without having to lay in a particular position as the convex outer curvature 32 of the crystal 18 will promote G-spot stimulation and the ribbing 20 will provide added sensations.

It should be understood, of course, that the foregoing relates to exemplary embodiments of the invention and that modifications may be made without departing from the spirit and scope of the invention as set forth in the following claims.

What is claimed is:

1. A sexual aid device, comprising:
a penile sleeve having an upper portion and a lower portion;
the upper portion dimensioned and adapted to be located along an upper portion of a human wearer's erect penis when said penile sleeve is worn;
the lower portion dimensioned and adapted to be located along a lower portion of a human wearer's erect penis when said penile sleeve is worn;
a plurality of stabilizing straps provided along the upper portion, each adjacent stabilizing strap are spaced apart by an upper void; and
a crystal opening provided in the lower portion.

2. The sexual aid device of claim 1, further comprising one or more crystal straps spanning the crystal opening.

3. The sexual aid device of claim 1, wherein each upper void is dimensioned for a human finger to pass therethrough.

4. The sexual aid device of claim 2, further comprising a plurality of ribbing disposed along the lower portion.

5. The sexual aid device of claim 2, further comprising a crystal dimensioned to be substantially coextensive with the crystal opening, wherein the crystal protrudes through the crystal opening.

6. The sexual aid device of claim 5, wherein the crystal provides a convex curvature that protrudes through the crystal opening.

7. The sexual aid device of claim 6, the crystal providing a concave curvature opposing the convex curvature, wherein the concave curvature supports the erect penis when the crystal is sandwiched between the one or more crystal straps and said erect penis.

8. A sexual aid device sexual aid device, comprising:
a penile sleeve having an upper portion and a lower portion;
the upper portion dimensioned and adapted to be located along an upper portion of a human wearer's erect penis when said penile sleeve is worn;
the lower portion dimensioned and adapted to be located along a lower portion of a human wearer's erect penis when said penile sleeve is worn;
a plurality of stabilizing straps provided along the upper portion, each adjacent stabilizing strap are spaced apart by an upper void, wherein each upper void is dimensioned for a human finger to pass therethrough;
a crystal opening provided in the lower portion;
one or more crystal straps spanning the crystal opening;
a plurality of ribbing disposed along the lower portion; and
a crystal dimensioned to be substantially coextensive with the crystal opening, wherein the crystal provides a convex curvature that protrudes through the crystal opening when the crystal is sandwiched between the one or more crystal straps and said erect penis.

9. The sexual aid device of claim 8, the crystal providing a concave curvature opposing the convex curvature, wherein the concave curvature supports the erect penis when the crystal is sandwiched between the one or more crystal straps and said erect penis.

10. A method of promoting sexual stimulation utilizing crystals during sexual intercourse, comprising:
providing the sexual aid device of claim 5; and
utilizing a finger through one of the upper voids to pull the penile sleeve over the erect penis so that the crystal is sandwich between both yet a portion of the crystal protrudes through the crystal opening.
